(19) United States
(12) Patent Application Publication
(10) Pub. No.: US 2022/0387721 A1
(43) Pub. Date: Dec. 8, 2022

(54) Device For Assisting In The Use Of A Device For Dispensing A Product

(71) Applicant: Nemera La Verpillière, LA VERPILLIERE (FR)

(72) Inventors: Thierry Decock, Lyon (FR); Lenaic Guimard, Saint-Cassien (FR); Adrien Delay, Vacquières (FR); Eric Poulain, Montluel (FR); Olivier Vernant, Anthon (FR)

(73) Assignee: Nemera La Verpillière, LA VERPILLIERE (FR)

(21) Appl. No.: 17/773,792

(22) PCT Filed: Oct. 30, 2020
(86) PCT No.: PCT/EP2020/080567

§ 371 Date: May 2, 2022

(30) Foreign Application Priority Data
Oct. 31, 2019 (FR) FR1912305

(51) Int. Cl. A61M 5/315; A61M 15/00

(52) U.S. Cl. CPC A61M 5/31565; A61M 15/0068; A61M 2205/3389

(57) ABSTRACT

An assistance device for the use with a product dispensing device includes an engaging unit to the dispensing device, a locking unit for locking the dispensing device and configured to prevent or permit dispensing of the product, and a measurement unit for measuring a quantity of the product dispensed by the dispensing device. The locking unit remains unlocked for a predetermined period of time starting from a predetermined activation of the assistance device and then locks automatically at the end of the predetermined period. The locking unit locks automatically when a quantity of the product dispensed during the predetermined period reaches or exceeds a predetermined threshold.

### FIELD OF THE INVENTION

The invention relates to an assistance device for the use of a product dispensing device, as well as a method for using a product dispensing device.

### BACKGROUND OF THE INVENTION

The state of the art, in particular document WO 2016/100564 already describes a assistance device for the use of a product dispensing device comprising locking means which prevent, after dispensing a complete dose of product, the dispensing of a second dose before the end of a time interval planned according to the posology of the medication. This prevents an overdose or the use of the device by a person other than the patient between the times planned for administration of the product. This is particularly advantageous for medications such as painkillers, in particular opioid-based painkillers.

Such a device has disadvantages. In particular, the assistance device is locked after delivering a complete dose of product throughout the period normally planned between taking the doses, which does not allow the patient to take a double dose of product, for example if the pain would be too great and not sufficiently relieved with the first dose. Furthermore, once the device is unlocked, product must be dispensed before the device can be relocked.

### SUMMARY OF THE INVENTION

The invention aims in particular to provide an assistance device for the use of a product dispensing device which overcomes at least some of these disadvantages.

Thus, the invention relates in particular to an assistance device for the use of a product dispensing device, the assistance device comprising:
engaging means to the dispensing device,
locking means for locking the dispensing device which are capable of preventing or permitting dispensing of the product, and
measurement means for measuring a quantity of product dispensed by the dispensing device,

the assistance device being arranged such that the locking means remain unlocked for a predetermined period of time starting from a predetermined activation of the dispensing device and then lock automatically at the end of the predetermined period of time, and in that it is further arranged such that the locking means lock automatically when a quantity of product dispensed during the predetermined period of time reaches or exceeds a predetermined threshold.

Thus, once the locking means have been unlocked, if no product is dispensed during the predetermined period of time, for example because finally the user does not want to obtain the product or because unlocking occurred accidentally, the locking means lock automatically, which avoids wasting product. Furthermore, this reduces the risk, if the locking means should be unlocked accidentally, of an unwanted quantity of product being dispensed.

A predetermined activation of the assistance device may, in particular, correspond to unlocking of the locking means. This unlocking may for example be carried out by authenticating a fingerprint using a fingerprint sensor or by entering a predetermined code.

In another possible embodiment, the assistance device can be activated by unlocking the locking means by voice authentication and/or a predetermined voice command emitted by the user.

The locking means are either locked to prevent product from being dispensed, or unlocked to permit dispensing of the product. In the locked position, the locking means lock the dispensing device which cannot be activated. In the unlocked position, the locking means unlock the dispensing device which can therefore be activated.

A predetermined activation of the dispensing device may in particular correspond to a complete activation of the dispensing device, i.e. an activation resulting in the dispensing of a maximum dose of product that can be delivered in a single time by the dispensing device. It may be for example a complete activation of a pump of the dispensing device. The predetermined activation could also correspond to a partial activation instead of a complete activation, corresponding for example to a predetermined stroke of an applicator of the dispensing device.

The threshold of quantity of product that can be dispensed during the predetermined period of time could correspond to a dose of product to be administered during a product intake. For example, for a medication, it is the dose that the patient must take during each medication intake. This dose could be reached by activating the dispensing device one or more times. Between the activations necessary to deliver a dose, the locking means do not lock automatically.

The fact that the locking means lock automatically if, during the predetermined period of time, a dispensed quantity of product reaches or exceeds the predetermined threshold reduces the risk of an overdose, especially if the product is a medication, preventing a dose greater than a maximum permitted dose of product from being delivered. Furthermore, this reduces the risk of wasting product if the dispensing device should be accidentally unlocked, for example in a bag or in a pocket, or if the dispensing device should be activated several times accidentally during the predetermined period of time. The dispensing device could therefore be activated only until the predetermined product threshold is reached rather than at the end of the predetermined period of time.

Advantageously, the predetermined period of time is between 1 and 20 minutes, preferably between 5 and 10 minutes.

This predetermined period of time is therefore long enough for the user, after unlocking the locking means, to activate the dispensing device, and short enough to keep the above-mentioned advantages relating to relocking after the predetermined period of time (reduction of the risk of overdose and of accidentally dispensing product).

Advantageously, the predetermined period of time being a first predetermined period of time, the assistance device further comprises control means capable of preventing or permitting unlocking of the locking means, the control means being configured to permit unlocking of the locking means during a second predetermined period of time starting from the automatic locking of the locking means,

the assistance device being arranged such that the locking means lock automatically at the end of the second period of time and such that the locking means lock automatically when a quantity of product dispensed during the second period of time reaches or exceeds the predetermined threshold.

Thus, the device gives the user sufficient time to check whether or not the product has had sufficient effect and gives the user the opportunity to unlock the locking means again to obtain a dose of product. This is particularly advantageous if the product is a painkiller type medication, for example an opioid-based painkiller, since the user can measure the effectiveness of the medication directly by the degree of pain relief felt. In this way, if the pain is greater than usual or if the first dose of medication did not provide sufficient pain relief, the assistance device allows the user to take at least a second dose during the second period of time. Once again, the risk that a third party for whom the product is not intended could use the dispensing device after the authorized user unlocked it is reduced since the locking means lock automatically at the end of the second period of time. In addition, since the locking means lock automatically when the predetermined product quantity threshold is reached or exceeded during the second period of time, the user cannot dispense doses of product an unlimited number of times after unlocking the locking means, which reduces the risk of an overdose.

Advantageously, the second period of time is between 1 and 60 minutes, preferably between 10 and 30 minutes.

This is the average time it takes for a medication such as a painkiller to take effect. This time is therefore long enough for the user to feel whether or not the product has taken effect and short enough to keep the above-mentioned advantages.

Advantageously, the control means are configured to prevent the locking means from unlocking during a third predetermined period of time starting from the automatic locking of the locking means at the end of the second period of time or when the quantity of product dispensed during the second period of time has reached or exceeded the predetermined threshold.

Thus, after delivering a second dose of product, the control means prevent the locking means from unlocking, and therefore from dispensing product, during the third period of time. This third period of time corresponds for example to the minimum time which must elapse between taking one dose (possibly a double dose) and the next. The risk of overdose is therefore once again reduced. This third period of time could be between one and eight hours, for example between two and six hours, or even between two and four hours.

Advantageously, the means for measuring the quantity of product dispensed comprise a counter for counting a number of predetermined activations of the dispensing device.

This is a simple way of measuring the quantity of product dispensed. If, for example, the dispensing device operates using a pump, activating the dispensing device dispenses a known quantity of product. Thus, the quantity of product dispensed can easily be deduced from the number of activations of the dispensing device.

In one embodiment, since the predetermined period of time is a first period of time, the assistance device further comprises control means capable of preventing or permitting unlocking of the locking means, the control means being configured to permit unlocking of the locking means during a second predetermined period of time starting from the automatic locking of the locking means, the assistance device being arranged such that the locking means lock automatically at the end of the second period of time and such that the locking means lock automatically when a quantity of product dispensed during the second period of time reaches or exceeds the predetermined threshold.

The second period of time could be between 1 and 60 minutes, preferably between 10 and 30 minutes.

The control means could be configured to prevent the locking means from unlocking during a third predetermined period of time starting from the automatic locking of the locking means after the second period of time or when the quantity of product dispensed during the second period of time has reached or exceeded the predetermined threshold.

In another configuration of the invention, the measurement means can include a sensor measuring the filling level of the dispensing device. If, for example, the dispensing device comprises a reservoir containing the product to be dispensed and if the initial product filling level is known, the filling level sensor can be used to determine the quantity of product at the time of the measurement. Thus, it is easy to deduce the quantity of product dispensed.

The means for measuring the quantity of product dispensed could comprise, as an alternative or in addition, other elements used to measure the quantity of product dispensed and which are known per se.

Similarly, in another embodiment of the invention, means for collecting and storing information and data relating to the user and his use of the dispensing device and/or of the assistance device can be associated with the measurement means. These data may, for example, include the number of activations of the dispensing device and/or the number of activations of the device. Lastly, these collection and storage means can be associated with communication means to transfer the information contained by the collection and storage means. Thus, these communication means may, for example, allow the health personnel and/or the user to retrieve information concerning the intake of product, if said product corresponds to a medical treatment.

Advantageously, the assistance device comprises signaling means configured to emit at least one signal providing information concerning the countdown of at least one of the first, second and third periods of time.

Thus, the user is better informed of the time left to take a first dose or an additional dose of product before the locking means lock or before the device can no longer be unlocked and is therefore not taken by surprise. Using the information concerning the third period of time, the user is also better informed of the time to wait before being able to unlock the device again to obtain a dose. The dispensing device is therefore simpler and safer to use. The signal could be audible and/or visual and/or vibratory. For example, the signal may consist of a countdown displayed on a screen of the assistance device and/or a voice speaking the remaining time and/or one or more vibrations of the assistance device that the user will have learned to associate with a given remaining time.

Advantageously, the assistance device comprises signaling means configured to emit at least one signal providing information concerning a number of activations of the dispensing device that are still authorized before the locking means automatically lock the dispensing device.

Thus, the user is better informed of the state of the dispensing device and the possibilities available. The dispensing device is therefore simpler and safer to use. The signal could be audible and/or visual and/or vibratory. For example, the signal may consist of a screen of the assistance device displaying the number of activations of the dispensing device that are still authorized and/or a voice speaking this number of activations of the device and/or one or more vibrations of the assistance device corresponding to this number of activations of the dispensing device. Furthermore, if the product is a medication, the treatment is easier to follow since the user knows the number of activations of the dispensing device required to obtain the quantity of product corresponding to the predetermined threshold, and when this threshold corresponds to the dose they must take each time, they are thus informed of the number of activations of the dispensing device required to obtain a complete dose.

The invention also relates to a method for using a dispensing device for dispensing a pharmaceutical or cosmetic product, comprising the following steps:
unlocking the dispensing device to permit dispensing of the product, and
automatic locking of the dispensing device, at the end a predetermined period of time starting from the unlocking of the dispensing device.

Automatic locking after the predetermined period of time prevents the dispensing device from being used until the next product intake, whether or not by the user for whom the product is intended, and therefore reduces the risk of overdose or accidental delivery of product, in particular when unlocking also occurred accidentally.

The predetermined period of time could be between 1 and 20 minutes, preferably between 5 and 10 minutes.

Advantageously, the dispensing device is automatically locked if a quantity of product dispensed during the predetermined period of time reaches or exceeds a predetermined threshold.

This reduces the risk of overdose, in particular if the product is a medication, by preventing a dose greater than the maximum permitted dose of product from being dispensed during the predetermined period of time. Furthermore, this reduces the risk of wasting product if the dispensing device should be accidentally unlocked, for example in a bag or in a pocket, or if the dispensing device should be activated several times accidentally during the predetermined period of time. The dispensing device could therefore be activated only until the predetermined product threshold is reached rather than at the end of the predetermined period of time.

### BRIEF DESCRIPTION OF THE DRAWINGS

We will now describe one embodiment of the invention given as a non-limiting example referring to the attached figures, on which:

FIG. 1A and FIG. 1B are perspective views of an assistance device according to the invention associated with a product dispensing device, in which a disposable part and a reusable part of the assembly formed by the assistance device and the dispensing device are shown respectively assembled and disassembled.

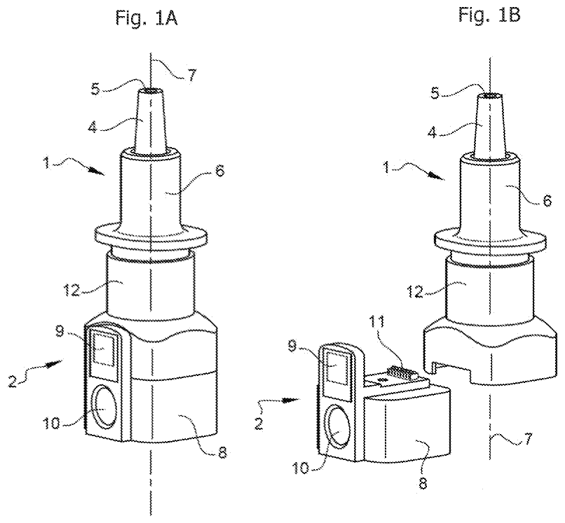

FIG. 2 is an exploded perspective view of a socket of the assistance device of FIG. 1A and FIG. 1B.

FIG. 3 is an exploded perspective view of the disposable part of the assistance device of FIG. 1A and FIG. 1B.

FIG. 4 is a diagrammatic representation of a method for registering the assistance device of FIG. 1A and FIG. 1B.

FIG. 5 is a diagrammatic representation of a method for configuring the assistance device of FIG. 1A and FIG. 1B.

FIG. 6 is a diagrammatic representation of a method for registering a fingerprint to unlock the assistance device of FIG. 1A and FIG. 1B.

FIG. 7 is a diagrammatic representation of a method for starting the assistance device of FIG. 1A and FIG. 1B.

FIG. 8 is a diagrammatic representation of an embodiment of the method for using a dispensing device according to the invention.

FIG. 9 is a diagrammatic representation of a method for replacing a disposable part of the assistance device of FIG. 1A and FIG. 1B.

### DETAILED DESCRIPTION OF THE INVENTION

The following embodiment describes an assistance device for the use of a product dispensing device, in which the dispensing device is a nasal spray and the product is a medication, in particular an opioid-based painkiller medication. The dispensing device could be any other dispensing device known by those skilled in the art, for example a device for dispensing liquid product in the form of drops. The product could also be any other product compatible for use with a device for dispensing a product, this product being a medication or not, for example a cosmetic product.

The dispensing device 1 to which the assistance device 2 is engaged comprises a pump 4 having a dispensing nozzle 5 and a reservoir 13. The dispensing device 1 further comprises a moving part forming an applicator 6. This applicator 6 is mounted movably so as to be able to slide along a main longitudinal axis 7 of the dispensing device 1 between a rest position, in which the applicator 6 is in its most distal position and therefore closest to the dispensing nozzle 5, and an activation position which is the minimum position that the applicator 6 must take so that the entire dose of product to be dispensed when the dispensing device 1 is activated is actually dispensed. It may be the most proximal position of the applicator 6, i.e. the farthest away from the dispensing nozzle 5. It could be another predetermined position, less proximal, i.e. nearer to the dispensing nozzle 5. Any other dispensing device could be used in association with the assistance device 2 according to the invention, provided that its operation is compatible with that of the assistance device 2.

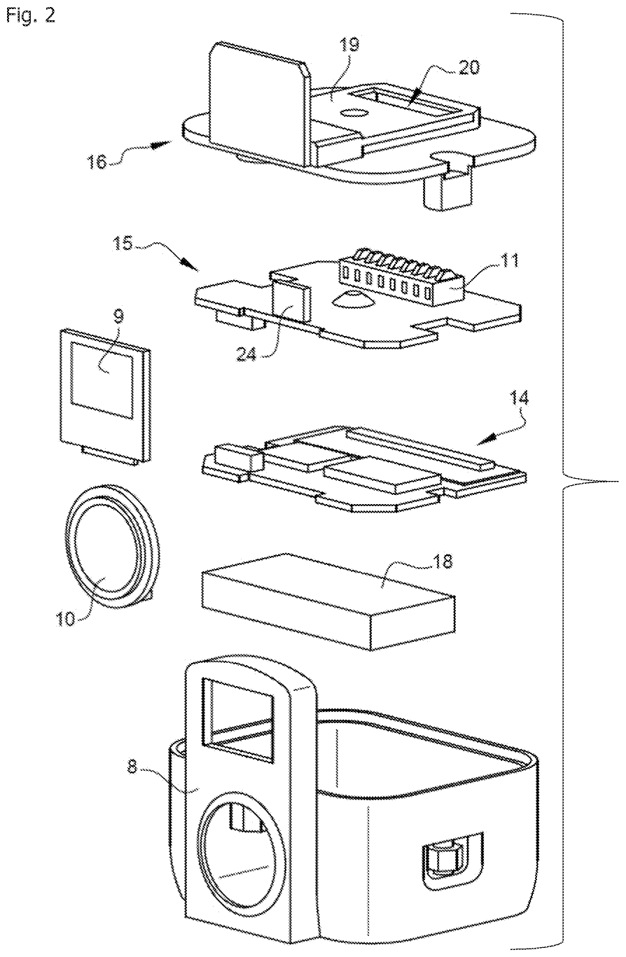

In this embodiment, the assistance device 2 is made of several parts. The assistance device 2 could nevertheless be made of one part. A first part is made of a reusable socket 8 (see FIG. 2). This socket 8 comprises in particular signaling means comprising a screen 9 capable of displaying a visual signal, a fingerprint sensor 10 and electronic components required to control and monitor the various functions of the assistance device 2. The electronic components of the socket 8 comprise electronic communication means comprising in the present case an electronic communication board 14 capable of receiving and transmitting data from and to a telecommunication network. Any other electronic communication means compatible with the operation of the assistance device 2 as described below could be used.

Another electronic component of the socket 8 is a main electronic board 15 capable of controlling and coordinating the various actions of the electronic components of the assistance device 2. The main electronic board 15 comprises an internal memory chip 24 capable of storing information. The socket 8 also includes a battery 18 capable of powering the various electronic components of the assistance device 2. The socket 8 comprises an upper wall 16. A relief 19 for connecting to the disposable part extends from this upper wall 16. This relief 19 has an orifice 20 though which an electronic pin 11 carried by the main electronic board 15 extends (see FIG. 2).

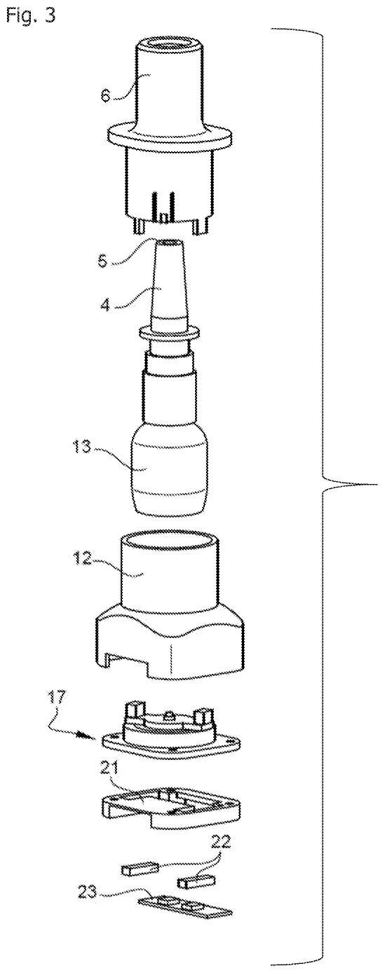

The assistance device 2 further comprises locking means 17 inside a base 12 (see FIG. 3). The base 12 comprises a lower wall 21 forming a support for the locking means 17 and has a lower face forming a cavity of shape complementary to that of the relief 19 extending from the socket 8 and capable of engaging with it in order to rigidly secure the base 12 and the socket 8. Connectors 22 and a disposable memory chip 23 also extend from the lower face of the lower wall 21 (see FIG. 3). When the disposable and reusable parts are assembled, the connectors 22 engage with the electronic pin 11 which allows them to communicate electronically with the main electronic board 15. Furthermore, the disposable memory chip 23 also engages with the electronic pin 11 and therefore communicates electronically with the main electronic board 15. The assistance device 2 also comprises engaging means (not shown) for rigidly securing to the dispensing device 1. The engaging means are known per se and may for example consist of clipping, insertion, screwing means, etc. The assistance device 2 comprises automated means for checking that at least one predetermined condition is met, the predetermined condition being independent of an interaction between firstly a user and secondly the assistance device 2 or the dispensing device 1 when delivering product to the user. The main electronic board 15 forms at least one part of the automated checking means. In the present case, these automated checking means are capable of checking that at least one of the conditions selected from priming the pump 4 of the dispensing device 1, an expiry date of the product, a concentration of the product, a state of use of the dispensing device 1, information identifying the assistance device, the product or the dispensing device 1 (batch or serial numbers not subjected to a market withdrawal request). These automated means could be capable of checking other predetermined conditions. The type of automated checking means depends on the predetermined condition to be checked. The automated checking means could for example comprise elements other than the main electronic board 15. These automated checking means could be configured to be implemented automatically when marketing the assistance device 2. This automatic implementation is controlled by the main electronic board 15.

The locking means 17 can be any locking means known by those skilled in the art and compatible with the operation of the dispensing device 1. These locking means 17 are capable of preventing or permitting dispensing of the product. In the present case, the locking means 17 consist of means movable between a locked position in which they prevent or limit a displacement of the applicator 6 of the dispensing device 1 and an unlocked position in which they do not prevent or limit a displacement of the applicator 6. The locking means 17 are moved from the locked position to the unlocked position and vice versa by a motor (not shown) controlled by the main electronic board 15. Any other means for moving the locking means 17 could be used, for example return means or a muscle wire.

The assistance device 2 also comprises measurement means for measuring a quantity of product dispensed. These measurement means are in particular capable of measuring a quantity of product dispensed during the period of time. These means may comprise for example measurement means for measuring a quantity of product in the reservoir 13. By comparing the quantity of product present in the reservoir 13 at two different times, these measurement means can be used to deduce the quantity of product dispensed between these two times. Other means for measuring the quantity of product dispensed known by those skilled in the art can be used. For example, the measurement means may comprise means for counting a number of activations of the dispensing device. If activation of the dispensing device 1 results in dispensing a predetermined quantity of product, for example if the dispensing device 1 comprises a pump 4, the number of activations of the dispensing device 1 can be used to deduce the quantity of product dispensed.

The assistance device 2 is arranged such that the locking means 17 remain unlocked for a predetermined period of time starting from a predetermined activation of the dispensing device 2 and then lock automatically at the end of the period of time, and is further arranged such that the locking means 17 lock automatically when a quantity of product dispensed during the predetermined period of time reaches or exceeds a predetermined threshold. This series of actions (holding in the unlocked position until the end of the period of time or until the quantity of product dispensed reaches or exceeds the threshold, then locking) is controlled by the main electronic board 15. In the present case, the predetermined activation of the assistance device 2 corresponds to unlocking of the locking means 17. This unlocking may for example be carried out by authenticating a fingerprint using a fingerprint sensor 10 or by entering a predetermined code. In the present case, the predetermined activation of the dispensing device 1 corresponds to a complete activation of the dispensing device, i.e. an activation resulting in the dispensing of a maximum dose of product that can be delivered in a single time by the dispensing device. This predetermined activation could correspond to an activation resulting in the dispensing of an incomplete dose of product, for example between 60% and 99% by volume of the maximum dose of product that can be delivered in a single activation. The assistance device 2 is configured such that the locking means 17 can be unlocked when the predetermined condition is met and remain locked when the predetermined condition is not met. Once again, this series of actions is controlled by the main electronic board 15 which thus forms control means capable of preventing or permitting unlocking of the locking means 17.

The above-mentioned period of time being a first period of time, the main electronic board 15 forming the control means is configured to permit unlocking of the locking means 17 during a second predetermined period of time starting from the automatic locking of the locking means 17. The assistance device 2 is arranged such that the locking means 17 lock automatically at the end of the second period of time and such that the locking means 17 lock automatically when a quantity of product dispensed during the second period of time reaches or exceeds the predetermined threshold.

The assistance device 2 comprises a time counter built into the main electronic board 15 which is capable of measuring the time elapsed since a given event. In particular, the time counter measures the time elapsed since the predetermined activation of the assistance device 2 and enables it to know when the first or second predetermined period of time has elapsed. The first period of time can be between 1 and 60 minutes, preferably between 20 and 30 minutes. In the present case, the period of time is 20 minutes. The second period of time can be between 1 and 20 minutes, preferably between 5 and 10 minutes. In the present case, the second period of time is 5 minutes.

The assistance device 2 could be configured such that the locking means 17 lock when the dispensing device 1 has been activated a predetermined number of times during the first period of time. The main board 15 collects the information concerning the number of activations made during the period of time and instructs the locking means 17 to lock when the predetermined number of times is reached during the period of time. This predetermined number of times corresponds to the quantity of product necessary to reach or exceed the predetermined threshold.

The main electronic board 15 forming the control means is configured to prevent the locking means 17 from unlocking during a third predetermined period of time starting from the automatic locking of the locking means after the second period of time or when the quantity of product dispensed during the second period of time has reached or exceeded the predetermined threshold. This third period of time can be measured by the above-mentioned time counter. In the present case, this third period of time is 4 hours.

The screen 9 of the signaling means is capable of emitting a signal providing information concerning the countdown of the first, second and third periods of time. The main electronic board 15 instructs the screen 9 to display the signal. When the locking means 17 are unlocked by the user, the main electronic board 15 instructs the screen 9 to display a timer indicating the time remaining before the locking means 17 lock automatically if a predetermined activation of the dispensing device 1 does not occur during the first period of time. This remaining time thus corresponds to the countdown of the first period of time. When the user performs the predetermined activation of the dispensing device 1, the main electronic board 15 sends an instruction to replace this period of time by a new period of time displaying the remaining time of the second period of time during which the main electronic board permits unlocking of the locking means 17. It therefore corresponds to the time remaining before the locking means 17 can no longer be unlocked until the expiry of the third period of time. The main electronic board 15 sends an instruction, at the end of the second period of time or, if the dispensing device 1 is activated during the second period of time, at the end of this activation, to display on the screen the remaining time of the third period of time. It therefore corresponds to the time remaining before the main electronic board 15 permits unlocking of the locking means. Once the locking means 17 have been locked, the screen 9 could display a message informing the user of this state. For example, the screen 9 can display a message such as “Device locked” or “Touch to unlock”.

In addition, the screen 9 of the signaling means is capable of emitting at least one signal providing the user with information concerning a number of activations of the dispensing device 1 that are still authorized before the locking means 17 automatically lock the dispensing device 1. Once again, the main electronic board 15 instructs the screen 9 to display the signal. This signal may simply consist in displaying on the screen 9 the number of activations still possible before the main electronic board 15 instructs the locking means 17 to lock the dispensing device 1.

Furthermore, the signaling means are capable of automatically emitting at least one signal when the predetermined condition is not met. The main electronic board 15 instructs the signaling means to emit the signal. This signal can be visual and/or audible and/or vibratory. For example, the signal could consist of a message displayed on the screen 9 and indicating the predetermined condition which is not met. It could also consist in speaking the condition which is not met, using sound generating means.

The electronic communication board 14 is capable of performing at least one of the steps comprising at least receiving information from a telecommunication network concerning a modification of the predetermined condition or of at least one of the predetermined conditions, transmitting information to the network concerning the predetermined condition or at least one of the predetermined conditions.

The final device, formed by the dispensing device 1 mounted on the assistance device 2 comprises a disposable part (see FIG. 3) and a reusable part (see FIG. 2). The reusable part is formed by the socket 8 of the assistance device 2 which comprises in particular the screen 9, the fingerprint sensor 10, the main electronic board 15, the electronic communication board 14 and the battery 18. The disposable part comprises the dispensing device 1 (the applicator 6, the pump 4 and the reservoir 13) as well as the base 12, the locking means 17, the disposable memory chip 23 and the connectors 22 of the assistance device 2. Savings can therefore be made with the reusable part which can be reused with other dispensing devices 1.

We will now describe the methods implemented when using the assistance device 2 according to the invention.

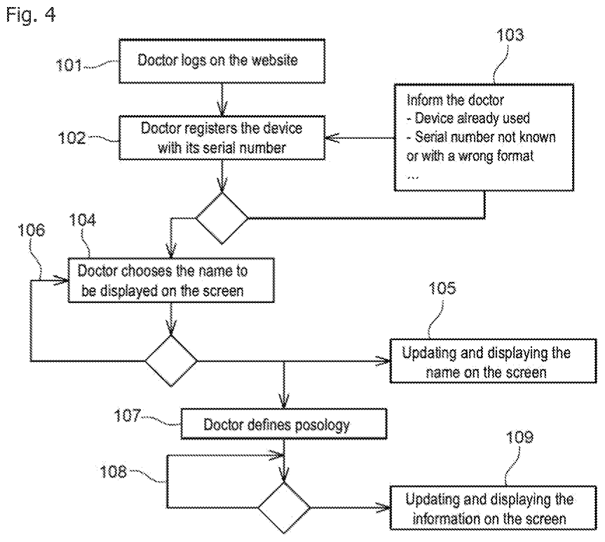

Firstly, the assistance device 2 is registered by a doctor, or any other person qualified to register it, on a telecommunication network provided for this purpose (see FIG. 4). To do this, the doctor logs on to this telecommunication network (step 101), for example a website, and registers the assistance device 2 by entering its serial number (step 102). If this serial number has already been registered, and is not known in a database listing the serial numbers of the various assistance devices 2 which have been produced or does not have the correct format, a message is displayed on a user interface of the network to inform the doctor who can then take the necessary measures (step 103). For example, the doctor may realize that they had not entered the correct serial number and enter it again or decide to use another assistance device 2. If the serial number of the assistance device 2 corresponds to a number subjected to a market withdrawal request, a message is displayed on the user interface to inform the doctor accordingly. This information could be transmitted by the telecommunication network to the assistance device 2 whose main electronic board 15 checks that the serial number of the assistance device 2 is actually present on this list and, consequently, the main electronic board 15 instructs the signaling means to emit a signal indicating that the condition relating to the absence of the assistance device 2 on a list of products subjected to a withdrawal request is not met. For example, the screen 9 displays “Assistance device withdrawn from the market”.

The doctor then chooses the name to be displayed on the screen 9 (step 104) for example when switching on the assistance device 2. This display is particularly advantageous to check, before administering the product, that it is really intended for the person to whom it is going to be administered. If the name entered is not accepted by the telecommunication network, for example because it does not correspond to a patient name in the database or because it contains characters not recognized by the telecommunication network, another name must be entered (step 106). If the name entered is accepted, the telecommunication network sends this information to the assistance device 2, its internal memory chip 24 stores this name and the main electronic board 15 instructs the screen 9 to display the name (step 105). The doctor or a person having the assistance device 2 can thus check that the name has been entered correctly.

Once the assistance device 2 has been registered on the telecommunication network and has been assigned a patient, the doctor can define at any time the posology of the treatment intended to be taken using the assistance device 2 (step 107). The doctor can in particular define the medication used as well as its dosage and the frequency of the intakes. The doctor can modify this posology whenever required (step 108). Each time a posology is defined, the information is transmitted to the assistance device 2 by the telecommunication network via the electronic communication board 14 and the main electronic board 15 instructs the screen 9 to display the new posology and the internal memory chip 24 to save this new posology (step 109).

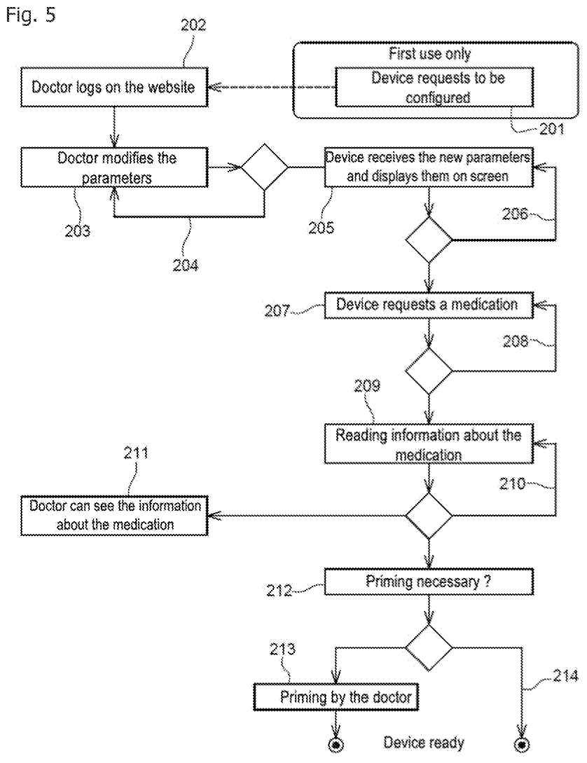

A method for configuring the assistance device 2 is described below (FIG. 5).

When the assistance device 2 is switched on for the first time, or if no configuration parameters have been associated with it, the main electronic board 15 instructs the electronic communication board 14 of the assistance device 2 to send a message requesting it to be configured (step 201). This message may consist of a message displayed on the screen 9 of the assistance device 2 or on a user interface of the telecommunication network. Following this message, the doctor configures or modifies the parameters (step 203) of the assistance device 2 by logging on, if necessary, to the telecommunication network (step 202). Configuring the assistance device 2 may include indicating the medication to be used with it, as well as its posology, whether the dosage or the frequency of the intakes. The doctor can also indicate whether or not the patient can take more than one dose at each intake, for example whether or not the patient can take a double dose. Once these data have been saved on the telecommunication network and if the assistance device 2 is connected to this network, these data are saved on the internal memory chip 24 of the assistance device 2 and the main electronic board 15 instructs the signaling means to display this new configuration on the screen 9 so that the person having the assistance device 2, for example the doctor or the patient, can read this information (step 205). The doctor can modify the configuration at any time (step 204). Once the device has been configured, the patient can use the device by unlocking it by touching the fingerprint sensor 10 with his finger. If the fingerprint associated with this assistance device 2 does not correspond to that of the patient, nothing happens (step 206). If the fingerprint corresponds, the main electronic board 15 instructs the screen 9 to display a message requesting the addition of the dispensing device 1 containing the medication (step 207). This message remains displayed until the dispensing device 1 containing the medication has been installed on the assistance device 2 (step 208). This installation can be carried out using methods known by those skilled in the art. The assistance device 2 comprises detection means capable of detecting the presence or absence of a dispensing device 1 installed on the assistance device 2. These detection means may consist of any means known per se. These detection means are capable of transmitting information to the main electronic board 15, this information indicating whether or not a dispensing device 1 is installed on the assistance device 2. Once the assistance device 2 has detected that a dispensing device 1 containing a medication has been installed, it reads the information of this dispensing device 1 (step 209), it being possible for example to read this information using the RFID (radio frequency identification) technology. For example, the dispensing device 1 could comprise an RFID tag containing information concerning the medication that the dispensing device 1 contains (active substance or trade name of the medication, concentration of the medication, quantity of product contained in the reservoir 13, etc.). This RFID tag is read by a reader included in the assistance device 2. The main electronic board 15 compares the information read on the RFID tag with the parameters entered by the doctor and which have been saved on the internal memory chip 24. If the information read on the RFID tag and the parameters saved on the internal memory chip 24 are different, i.e. the predetermined conditions concerning these parameters are not met, the assistance device 2 returns to the previous step (step 210) and the main electronic board 15 instructs the signaling means to display a message on the screen 9 to inform the user that the medication associated with the assistance device 2 does not have at least one of the required characteristics. A warning message could be sent to the doctor via the telecommunication network. If the information read on the RFID tag and the parameters saved correspond, a message is sent on the telecommunication network and can be read by the doctor so that the doctor can see the information concerning the new medication installed on the assistance device 2 (step 211). The RFID tag also comprises information concerning another predetermined condition as to whether or not the dispensing device 1 containing the medication should be primed, in particular when this dispensing device 1 comprises a pump 4. The predetermined condition to be met is therefore that the pump 4 of the dispensing device 1 has been primed. If priming is required, i.e. the corresponding predetermined condition is not met, the main electronic board 15 instructs the screen 9 to display a message to inform the user accordingly. The message could also indicate the person to be contacted by the user to carry out the priming, for example the doctor (step 213). If no priming is required, either no message is displayed on the screen 9, or a message indicating that the dispensing device 1 is ready to be used is displayed on the screen 9 (step 214).

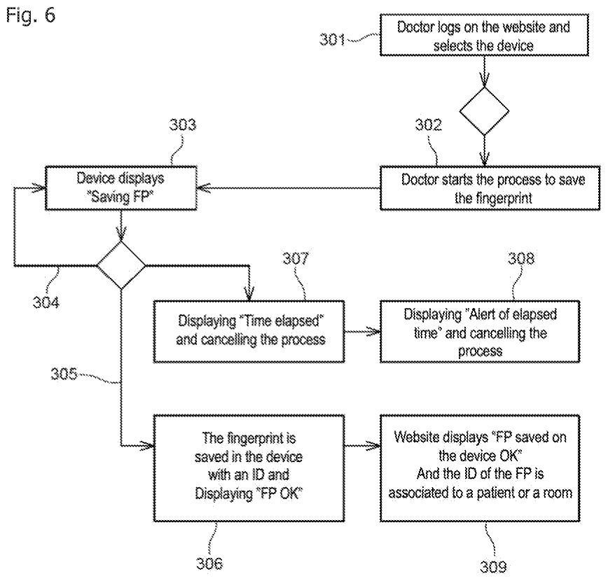

If the device is unlocked by authenticating a fingerprint using the fingerprint sensor 10, this fingerprint must first be registered (see FIG. 6).

To do this, the doctor logs on to the same telecommunication network as before and selects the assistance device 2 to be associated with a fingerprint (step 301). The doctor can search for the assistance device 2 in the database either using its serial number or the patient's name which has already been saved and associated with the assistance device 2, according to the method described previously.

If the assistance device 2 is not connected to the telecommunication network, a message informs the doctor that they cannot connect to the assistance device 2. In this case, the doctor can either switch on the assistance device 2 so that it can connect to the telecommunication network, or wait until another person, for example the patient, switches it on. Once the assistance device 2 is connected to the telecommunication network, the doctor starts the process to save the fingerprint (step 302). The main electronic board 15 then instructs the screen 9 to display “Saving FP” (step 303) and the patient places one of their fingers, for example an index finger, on the fingerprint sensor 10.

If the assistance device 2 cannot manage to save the fingerprint correctly, for example if the patient has moved their finger when saving, the screen 9 continues to display the same message (step 304). If no fingerprint could be saved correctly during a given period of time, for example one minute, the main electronic board 15 instructs the signaling means to display on the screen 9 a message to inform the patient, for example “Time elapsed” and the fingerprint saving procedure is cancelled (step 307). A warning message is also sent on the telecommunication network, in particular to inform the doctor (step 308). This message may be “Alert of elapsed time”.

If a fingerprint is saved correctly during the given time, an identification number is associated with this fingerprint (fingerprint ID) and the main electronic board 15 associates it with an identifier of the patient to whom it corresponds, for example a patient ID or the patient's name, and instructs the electronic communication board 14 to send a message to the telecommunication network so that this information (fingerprint ID and patient ID) is saved (steps 306 and 309). Furthermore, the screen 9 then displays the message “FP OK”. On a user interface of the telecommunication network, the message “FP saved on the device OK” is displayed to inform the doctor that the patient's fingerprint has been correctly saved and associated with the assistance device 2. The information is then saved in the database associating the assistance device 2 with the patient. Since the data concerning the patient's fingerprint are saved in the internal memory chip 24 of the assistance device 2, the patient can unlock the locking means 17 even when the assistance device 2 is not connected to the telecommunication network.

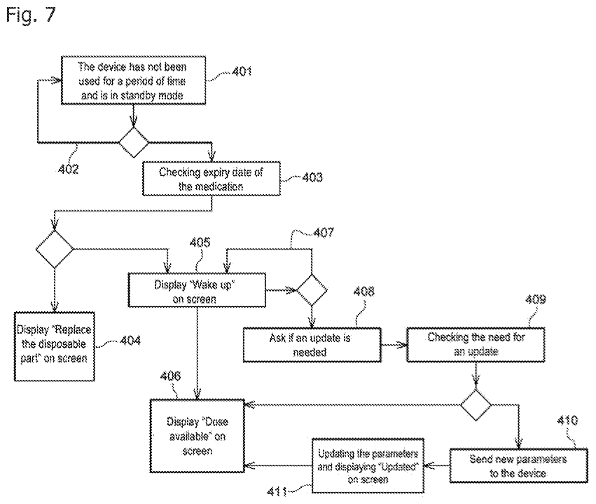

We will now describe a method for exiting a standby mode of the assistance device 2 (see FIG. 7).

If the assistance device 2 has not been used for a prolonged period of time, for example several hours, the assistance device 2 switches to standby mode (step 401). It is necessary to exit this standby mode in order to use the dispensing device 1 associated with the assistance device 2. To do this, the user presses his finger whose fingerprint has been saved on the fingerprint sensor 10. Control means connected to the main electronic board 15 check that the fingerprint detected by the fingerprint sensor 10 corresponds to the identifier number associated with the fingerprint saved on the internal memory chip 24 and therefore corresponds to a person authorized to use the dispensing device 1 associated with the assistance device 2. If the fingerprint does not correspond to a fingerprint saved, the assistance device 2 remains in standby mode and the locking means 17 of the assistance device 2 remain locked (step 402). If the fingerprint corresponds to at least one fingerprint saved, the main electronic board 15 checks whether a predetermined date, for example an expiry date of the product contained in the dispensing device 1, has been exceeded (step 403). The expiry date could be indicated on the RFID tag which is read by the assistance device 2. If the expiry date is exceeded, the main electronic board 15 instructs the screen 9 to display a message prompting to replace the disposable part, the message indicating for example “Replace the disposable part” (step 404). If the expiry date is not exceeded, the main electronic board 15 instructs the screen 9 to display a message indicating that the assistance device 2 is exiting standby mode, for example the screen 9 displays “Wake up” (step 405). The assistance device 2 then either displays a message on the screen 9 to indicate that the dispensing device 1 is ready to be used, for example a message indicating “Dose available” or “Ready” and the main electronic board 15 sends an instruction to unlock the locking means 17 (step 406), or the assistance device 2 tries to access the telecommunication network to check whether or not updates of the parameters of the assistance device 2 are available (step 408). In the latter case, the assistance device 2 tries to access the telecommunication network and displays “Wake up” on the screen 9 until it manages to do so (step 407). Once it has managed, control means check whether or not the assistance device 2 needs to be updated (step 409). If no update is required, the dispensing device 1 is ready to be used and the main electronic board 15 instructs the screen 9 to display a message accordingly, for example “Dose available” or “Ready” (step 406), and the main electronic board 15 sends an instruction to unlock the locking means 17. If an update is required, for example an update of a configuration parameter of the assistance device 2 such as a new posology, the telecommunication means send the data including these new parameters to the assistance device 2 and the main electronic board 15 instructs the internal memory chip 24 to save these data (step 410). The screen 9 then displays a message indicating that the assistance device 2 has been updated, for example the screen 9 displays “Updated” (step 411). The main electronic board 15 then instructs the screen 9 to display a message indicating that the dispensing device 1 is ready to be used, for example “Dose available” or “Ready” and sends an instruction to unlock the locking means 17 (step 406).

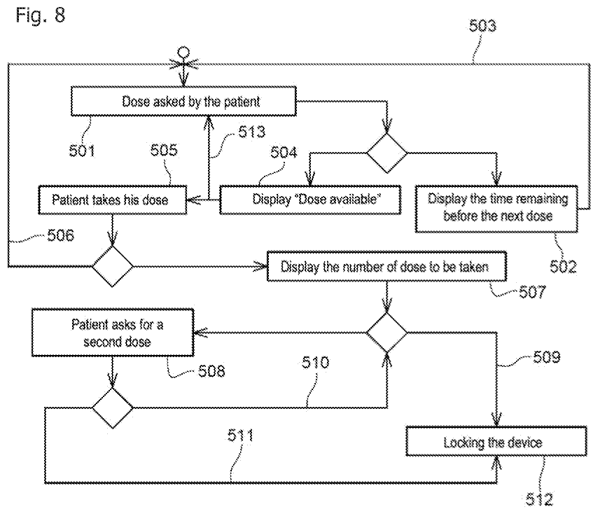

We will now describe a method for using the dispensing device 1 according to the invention (see FIG. 8).

In this example, the product is a painkiller medication, the patient therefore uses the assistance device 2 and the dispensing device 1 when he requires pain relief. To unlock the device, the patient presses his fingerprint on the fingerprint sensor 10 after having saved the fingerprint according to the method described previously (step 501). Firstly, the assistance device 2 checks that the patient is authorized to take a dose according to the characteristics entered by the doctor when configuring the device. In particular, the main electronic board 15 checks that the third period of time has expired. If the patient, in view of the configuration made by the doctor, is not authorized to take a dose at this time, for example because the third period of time has not expired, the main electronic board 15 prevents the locking means 17 from unlocking and instructs the screen 9 to display the time remaining before the patient can take a dose, i.e. the remaining time of the third period of time (steps 502 and 503). In addition, if the patient's fingerprint does not correspond to that saved previously, the main electronic board 15 then also instructs the locking means 17 to remain locked. The main electronic board 15 could instruct the screen 9 to display a message informing the patient that the fingerprint does not correspond to the fingerprint saved to inform them of the reason why the locking means 17 do not unlock. If the patient's fingerprint corresponds and the posology saved by the doctor allows the patient to take medication, for example if the third period of time has expired, the main electronic board 15 instructs the locking means 17 to unlock and instructs the screen 9 to display a message indicating that the dose can be taken, for example “Dose available” (step 504).

Once the locking means 17 are unlocked, the predetermined activation of the dispensing device 1 is possible. The timer counts down the first period of time starting from this unlocking. If the period of time measured by the timer reaches or exceeds the first predetermined period of time and the predetermined activation has not occurred, the main electronic board 15 instructs the locking means 17 to lock. In the present case, the first predetermined period of time is 5 minutes.

Thus, the patient must take a first dose of medication during the first predetermined period of time to prevent the locking means 17 from relocking (step 513). To do this, after correctly positioning the dispensing device 1 (in this case, since it is a nasal spray, the patient introduces the dispensing nozzle 5 in one of his nostrils), the patient activates the dispensing device 1 according to the predetermined activation (step 505). As indicated previously, in the present case, this predetermined activation consists of an activation which results in dispensing a maximum dose of product that can be delivered in a single time by the dispensing device 1. After dispensing this first dose of medication, the main electronic board 15 instructs the locking means 17 to lock and instructs the screen 9 to display the number of doses that can still be taken as well as the remaining time of the second period of time during which the user can unlock the locking means 17 again to obtain a second dose of product (step 507). Thus, during this period, the patient can determine whether or not an additional dose is required to relieve the pain felt. After the second period of time, the main electronic board 15 prevents the locking means 17 from unlocking until the third period of time has expired (step 509). To obtain a second dose, the patient unlocks the locking means 17 in the same way as described previously (step 508) and activates the dispensing device 1 to obtain the dose. Once the dose has been dispensed, the main electronic board 15 instructs the locking means 17 to lock (step 512). The patient could request more than one additional dose during the second period of time (step 510). In this case, once the second dose of product has been dispensed and the locking means 17 locked, the patient can unlock the locking means 17 again in the same way as described previously, provided that the second period of time has not yet expired.

In the present case, the assistance device 2 is configured such that the locking means 17 lock when the dispensing device 1 has been activated once during the first period of time, which means that the quantity of product dispensed when activating the dispensing device 1 reaches or exceeds the predetermined threshold, i.e. it corresponds to the dose to be taken by the patient during each intake of medication. This dose could be reached by activating the dispensing device 1 several times.

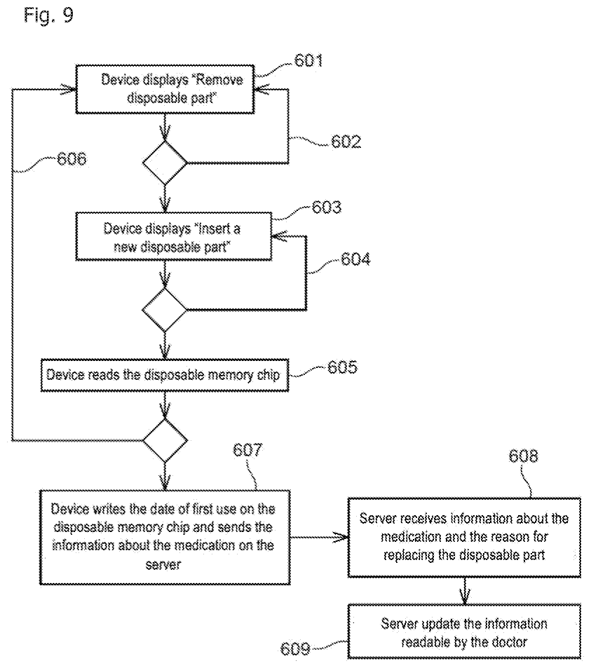

We will now describe a method for replacing the disposable part of the assembly formed by the assistance device 2 and the dispensing device 1 (FIG. 9).

As indicated previously, the disposable part comprises the dispensing device 1 as well as the base 12 and the locking means 17 of the assistance device 2. There are several situations in which it is recommended to replace the disposable part, in particular when the reservoir 13 of the dispensing device 1 is empty, when another product (for example another medication) must be used, or when the product has exceeded a predetermined date, for example an expiry date.

The assistance device 2 is configured to detect the cases where the disposable part should be replaced. This detection can be made by the main electronic board 15 or another element of the automated means for checking a predetermined condition. For example and as indicated previously, the assistance device 2 is capable of reading information present on the RFID tag of the dispensing device 1 concerning the expiry date of the product contained in the reservoir 13. Furthermore, the assistance device 2 may comprise sensors capable of measuring a filling level of the reservoir 13 of the dispensing device 1. Thus, when the device detects a situation where the disposable part should be replaced, the main electronic board 15 instructs the screen 9 of the signaling means to display a message informing that this replacement must be carried out, for example the screen 9 displays “Remove disposable part” (step 601). The assistance device 2 is configured to display this message until the disposable part is separated from the reusable part (step 602). Once the disposable part has been removed, the main electronic board 15 instructs the screen 9 to display a message prompting to insert a new disposable part, for example the screen 9 displays “Insert a new disposable part” (step 603). This message is displayed until the detector used to detect the presence of a disposable part detects the presence of a disposable part associated with the reusable part (step 604). Once a new disposable part has been inserted, the assistance device 2 reads the disposable memory chip 23 and the automated checking means check that at least two conditions are met (step 605). These actions are controlled by the main electronic board 15 which forms at least one part of the automated checking means. The first condition is that the disposable part added is new, i.e. it has not yet been used in association with a reusable part. The automated checking means could check only whether or not the dispensing device 1 of the disposable part is new. This information can be checked by reading the memory chip 23 since it saves the data related to a first use of the disposable part. The second condition is that the concentration of the medication contained in the reservoir 13 corresponds to the parameters indicated by the doctor when configuring the assistance device 2. If at least one of these conditions is not met, the main electronic board 15 instructs the screen 9 to display a message prompting to remove the disposable part, for example “Remove the disposable part” (step 606). If at least the two conditions are met, the main electronic board 15 sends an instruction to write the date of first use on the disposable memory chip 23 present in the disposable part and the electronic communication board 14 sends the information concerning the medication present in the dispensing device 1 to the telecommunication network (step 607). The telecommunication network receives and saves the information concerning the new medication and the reasons for replacing the disposable part (step 608). This information is updated and can be read by the doctor on the telecommunication network (step 609). The doctor is therefore better informed of how the treatment is followed by the patient.

The invention is not limited to the embodiment described and other embodiments will be clearly apparent to those skilled in the art.

1. An assistance device for use of with a product dispensing device, the assistance device comprising:
an engaging until to the dispensing device,
a locking unit configured to lock the dispensing device and prevent or permit dispensing of the product, and
a measurement unit configured to measure a quanity of the product dispensed by the dispensing device,
wherein the locking unit remains unlocked for a predetermined period of time starting from a predetermined activation of the dispensing device and then lock automatically at the end of the predetermined period of time, and
wherein the locking unit locks automatically when a quantity of the product dispensed during the predetermined period of time reaches or exceeds a predetermined threshold.

2.The assistance device according to claim 1, wherein the predetermined period of time is between 1 and 30 minutes, preferably between 5 and 10 minutes.

3. The assistance device according to claim 1, wherein the predetermined period of time is a first period of time, and wherein the assistance device further comprises a control unit configured to prevent or permit unlocking of the locking unit, the control unit configured to permit unlocking of the locking unit during a second predetermined period of time starting from the automatic locking of the locking unit, the assistance device arranged such that the locking unit locks automatically at the end of the second predetermined period of time and such that the locking unit locks automatically when a quantity of the product dispensed during the second predetermined period of time reaches or exceeds the predetermined threshold.

4. The assistance device according to claim 3, wherein the second predetermined period of time is between 1 and 60 minutes, preferably between 10 and 30 minutes.

5. The assistance device according to claim 3, wherein the control unit is configured to prevent the locking unit from unlocking during a third predetermined period of time starting from the automatic locking of the locking unit 4at the end of the second predetermined period of time or when the quantity of the product dispensed during the second predetermined period of time has reached or exceeded the predetermined threshold.

6. The assistance device according to claim 1, wherein the measurement unit for measuring the quantity of the product dispensed comprises a counter for counting a number of predetermined activations of the dispensing device.

7. The assistance device according to claim 5, further comprising signaling unit configured to emit at least one signal providing information concerning the countdown of at least one of the first, second and third predetermined periods of time.

8. The assistance device according to claim 1, fuerther comprising signaling unit configured to emit at least one signal providing information concerning a number of activations of the dispensing device that are still authorized before the locking unit automatically lock the dispensing device.

9. A method for using a dispensing device for dispensing a pharmaceutical or cosmetic product, comprising the following steps:
unlocking the dispensing device to permit dispensing of the product, and
automatically locking the dispensing device at the end of a predetermined period of time starting from the unlocking of the dispensing device.

10. The method according to claim 9, wherein the dispensing device is automatically locked if a quantity of product dispensed during the predetermined period of time reaches or exceeds a predetermined threshold.
